(19) United States
(12) Patent Application Publication
(10) Pub. No.: US 2023/0270686 A1
(43) Pub. Date: Aug. 31, 2023

(54) RECOMBINANT GAS VESICLE NANOPARTICLES

(71) Applicant: PUKYONG NATIONAL UNIVERSITY INDUSTRY UNIVERSITY COOPERATION FOUNDATION, Busan (KR)

(72) Inventors: Jong-Myoung KIM, Busan (KR); Shiladitya DASSARMA, Baltimore, MD (US); Priya DASSARMA, Baltimore, MD (US)

(73) Assignee: PUKYONG NATIONAL UNIVERSITY INDUSTRY UNIVERSITY COOPERATION FOUNDATION, Busan (KR)

(21) Appl. No.: 18/055,177

(22) Filed: Nov. 14, 2022

(30) Foreign Application Priority Data
Dec. 21, 2021 (KR) 10-2021-0184203

(51) Int. Cl. A61K 9/51; C07K 16/18; C07K 14/215

(52) U.S. Cl. CPC A61K 9/5169; C07K 16/18; C07K 14/215; B82Y 5/00

(57) ABSTRACT

The present disclosure relates to a recombinant gas vesicle nanoparticle, and the recombinant gas vesicle nanoparticle prepared by the method of the present disclosure is a nano-sized protein particle that it is biologically non-toxic and safe. Further, its outer surface protein, GvpC, can be recombined to include the exogenous protein to reconstitute gas vesicle nanoparticles presenting/mounting the exogenous protein on the surface, so that it can be used as various antigen-loaded vaccine compositions that are active under physiological conditions, therapeutic target-specific carriers, including a sensor for target-specific therapy.

### CROSS-REFERENCE TO RELATED APPLICATIONS

This application is based on and claims priority from Korean Patent Application No. 10-2021-0184203, filed on Dec. 21, 2021, with the Korean Intellectual Property Office, the disclosure of which is incorporated herein in its entirety by reference.

### SEQUENCE LISTING

The content of the electronically submitted sequence listing, file name: Q281843_sequence listing as filed.XML; size: 15,827 bytes; and date of creation: Nov. 13, 2022, filed herewith, is incorporated herein by reference in its entirety.

### TECHNICAL FIELD

The present disclosure relates to recombinant gas vesicle nanoparticles (GVNPs).

### BACKGROUND

Gas vesicle nanoparticles (GVNPs) are found in cyanobacteria and Archaea and are very small, hollow structures that can be filled with gas. GVNPs are also called gas vesicles (GVs) and have a size of several tens to hundreds of nm, promoting cell flotation in water to increase the availability to light and oxygen, thereby improving photosynthetic or phototrophic capacity to favor cell survival in aquatic environments (DasSarma and DasSarma 2021). GVNPs, which provide buoyancy to cells by excluding water by hydrophobicity of the inner surface of the structure, form of a cylindrical rod mid-section and both ends are closed with a conical cap. Two proteins called GvpA and GvpC are known as major components of gas vesicles. Among them, GvpA is a major component of gas vesicles composed of about 70 amino acids and forms the core of the structure, with GvpC, 382 amino acids, which forming a complex with GvpA.

It was found that in Halobacterium sp. NRC-1 a halophilic archaeon (haloarchaeon) model system, a gene cluster encoding 12 proteins or more (gvpMLKJIHGFEDACNO) is involved in GVNP biogenesis (Jones et al. 1991; DasSarma and Arora 1997; DasSarma and DasSarma 2021). In addition to analyzing the GV-deficient phenotype according to each gyp gene mutation (DasSarma et al. 1994), seven gyp gene products were found to exist, including the GvpA protein, which is composed of the GV membrane, which is the main backbone of the gas vesicle protein complex, the GvpC protein which promotes GV growth, plays a structural reinforcing role, and is located outside, and five other gene products of unknown function such as GvpF, GvpG, GvpJ, GvpL and GvpM (DasSarma et al. 1987; Halladay et al. 1993; Shukla and DasSarma 2004; Chu et al. 2011). It is known that among them, GvpC present on the GV surface is a highly acidic (pI 3.57) hydrophilic protein with an expected molecular weight of 42,391 and stabilizes nanoparticles at high salinity occurring in the cytoplasm of extremely halophiles in Halobacterium sp. NRC-1.

### SUMMARY

The present disclosure has been made in an effort to provide a method for preparing a recombinant gas vesicle nanoparticle (GVNP).

Further, the present disclosure has been made in an effort to provide a recombinant gas vesicle nanoparticle.

Further, the present disclosure has been made in an effort to provide a vaccine composition.

Further, the present disclosure has been made in an effort to provide an antibody-loaded gas vesicle nanoparticle.

Further, the present disclosure has been made in an effort to provide a pharmaceutical composition for preventing or treating a disease or disorder.

An embodiment of the present disclosure provides a method for preparing a recombinant gas vesicle nanoparticle

Another embodiment of the present disclosure provides a recombinant gas vesicle nanoparticle prepared by the method.

Yet another embodiment of the present disclosure provides a vaccine composition including the recombinant gas vesicle nanoparticle.

Still another embodiment of the present disclosure provides an antibody-loaded gas vesicle nanoparticle.

Further, another embodiment of the present disclosure provides a pharmaceutical composition for preventing or treating a disease or disorder, the composition including the antibody-loaded gas vesicle nanoparticle as an active ingredient.

According to the embodiments of the present disclosure, recombinant gas vesicle nanoparticles prepared by the method of the present disclosure are nano-sized protein particles and have no associated nucleic acids or genes and that they are biologically non-toxic and safe. Further, their outer surface protein, GvpC, can be genetically modified to include the exogenous protein to reconstitute gas vesicle nanoparticles presenting/mounting the exogenous protein on the surface so that they can be used as various antigen-loaded vaccine compositions that are active under physiological conditions, therapeutic target-specific carriers, including a sensor for target-specific therapy.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE DRAWINGS

FIG. 1 is a schematic view of constructs including recombinant GvpC-IgG-binding (GB) domain fusion proteins GvpC4 GB and GvpC3 GB, respectively, and a diagram confirming its expression by immunoblot analysis;

FIG. 2 is a view confirming the production efficiency of the GVNP complex to which the fusion protein is bound under various conditions;

a) of FIG. 3 is a view confirming the formation of the GVNP complex according to the mixing ratio of the fusion proteins GvpC3 GB and GvpC4 GB and b) of FIG. 3 is a view confirming the binding stability of the fusion protein to GVNP:
Lane 1: subnatant of GVNP complex in which GvpC3 GB and GvpC4 GB were mixed in a ratio of 5:1;
Lane 2: top fraction of the GVNP complex in which GvpC3 GB and GvpC4 GB were mixed in a ratio of 5:1;
Lane 3: subnatant of GVNP complex in which GvpC3 GB and GvpC4 GB were mixed in a ratio of 1:1;
Lane 4: top fraction of the GVNP complex in which GvpC3 GB and GvpC4 GB were mixed in a ratio of 1:1;
Lane 5: subnatant of GVNP complex in which GvpC3 GB and GvpC4 GB were mixed in a ratio of 1:5; and
Lane 6: top fraction of the GVNP complex in which GvpC3 GB and GvpC4 GB were mixed in a ratio of 1:5;

FIG. 4 is a diagram confirming the functionality of the GVNP complex of which recombinant GvpCGB is bound to the surface:
A: Detection using chemiluminescence reaction after GvpC3 GB and GVNP reaction under various salt concentration conditions and then GVNP layer separation by centrifugation:
1: subnatant under 1× PBS condition;
2: top fraction under 1×PBS condition;
3: subnatant under 3 M NaCl condition; and
4: top fraction under 3 M NaCl conditions;
B: Detection using a chemiluminescence reaction in a mixture of GVNP and HRP-linked secondary antibodies in the presence or absence of GvpC3 GB:
1: subnatant of GVNPs+HRP-linked anti-rabbit antibodies mixture;
2: top fraction of GVNPs+HRP-linked anti-rabbit antibodies mixture;
3: subnatant of GVNPs+HRP-linked anti-rabbit antibodies+GvpC3 GB mixture; and
4: top fraction of GVNPs+HRP-linked anti-rabbit antibodies+GvpC3 GB mixture; and

FIG. 5 is a schematic diagram illustrating the process of binding an exogenously generated GvpCGB fusion protein in the present disclosure to GVNPs to construct a GVNP complex and binding IgG to the external surface thereof.

### DETAILED DESCRIPTION

In the following detailed description, reference is made to the accompanying drawing, which forms a part hereof. The illustrative embodiments described in the detailed description, drawing, and claims are not meant to be limiting. Other embodiments may be utilized, and other changes may be made, without departing from the spirit or scope of the subject matter presented here.

Hereinafter, the present disclosure is described in detail as an embodiment of the present disclosure with reference to the accompanying drawings. However, the following embodiment is presented as an example for the present disclosure, and if it is determined that a detailed description of a well-known technology or configuration known to those skilled in the art may unnecessarily obscure the gist of the present disclosure, the detailed description may be omitted, and the present disclosure is not limited thereby. The present disclosure may be variously modified and applied within the scope of the description and equivalents interpreted there from of the following claims.

Further, the terms used in this specification are terms used to properly express a preferred embodiment of the present disclosure, which may vary depending on the intention of a user or operator, or a custom in the field to which the present disclosure belongs. Accordingly, definitions of these terms should be made based on the content throughout this specification. Throughout the specification, when a part “includes” a certain element, it means that other elements may be further included, rather than excluding other elements, unless otherwise stated.

In one aspect, the present disclosure relates to a method for preparing a recombinant gas vesicle nanoparticle (GVNP), the method including steps of a) culturing Halobacterium sp.; b) collecting a gas vesicle (GV); c) removing GvpC from the gas vesicle; d) preparing a recombinant protein as an exogenous recombinant protein in which the full length or fragment of GvpC and an exogenous protein are fused; and e) reacting the gas vesicle from which GvpC has been removed with the exogenous recombinant protein to form a recombinant GVNP complex.

In one embodiment, the Halobacterium sp. may be Halobacterium sp NRC-1.

In one embodiment, the GvpC may be removed by treatment with urea and dialysis in step c).

In one embodiment, the full length of GvpC may include the amino acid sequence represented by SEQ ID NO: 1, and the fragment of GvpC may include the amino acid sequence represented by SEQ ID NO: 2.

In one embodiment, the full length of GvpC may be encoded by the nucleotide sequence represented by SEQ ID NO: 6, and the fragment of GvpC may be encoded by the nucleotide sequence represented by SEQ ID NO: 7.

In one embodiment, the exogenous protein may be at least one antigen, and the antigen may be a viral epitope, more preferably a fish virus epitope.

In one embodiment, the exogenous protein may be an IgG-binding (GB) domain, in which case the method may further include a step of binding at least one antibody to the recombinant GVNP complex.

In one embodiment, the IgG-binding (GB) domain may include the amino acid sequence represented by SEQ ID NO: 3 and may be encoded by the nucleotide sequence represented by SEQ ID NO: 8.

In one embodiment, the recombinant protein may be a recombinant protein in which the full length of GvpC and the IgG-binding (GB) domain are fused, may include the amino acid sequence represented by SEQ ID NO: 4, and may be encoded by the nucleotide sequence represented by SEQ ID NO: 9.

In one embodiment, the recombinant protein may be a recombinant protein in which a fragment of GvpC and the IgG-binding (GB) domain are fused, may include the amino acid sequence represented by SEQ ID NO: 5, and may be encoded by the nucleotide sequence represented by SEQ ID NO: 10.

In one embodiment, the exogenous recombinant protein may be expressed and purified in E. coli.

In one embodiment, the gas vesicle (GVNP) from which GvpC has been removed in step c) and the recombinant GvpCGB prepared in step d) are mixed in 1× PBS containing 3 M NaCl and reacted at room temperature for 2 hours. After centrifugation at low speed (60× g), the collected supernatant is mixed with 1× PBS solution containing 3 M NaCl, and then the above method is repeated to form the recombinant GVNP complex (GvpCGB::GVNP). Then, it is isolated and purified, and the target antibody is mixed with the isolated and purified GVNP complex. The above process can be repeated to prepare the antibody-binding GVNP.

In one embodiment, the antibody may be a polyclonal antibody, a monoclonal antibody, a minibody, a domain antibody, a bispecific antibody, an antibody mimic, a chimeric antibody, an antibody conjugate, a human antibody or a humanized antibody, or a fragment thereof. The fragment may be any one selected from the group consisting of Fab, Fd, Fab′, dAb, F(ab′), F(ab′)2, single chain fragment variable (scFv), Fv, single chain antibody, Fv dimer, complementarity determining region fragment, humanized antibody, a chimeric antibody and a diabody.

The antibody includes not only the form of a whole antibody, but also includes functional fragments of antibody molecules. The whole antibody has a structure having two full-length light chains and two full-length heavy chains, and each light chain is connected to the heavy chain by a disulfide bond. A functional fragment of an antibody molecule refers to a fragment having an antigen-binding function, and examples of antibody fragments include (i) an Fab fragment consisting of a light chain variable region (VL), a heavy chain variable region (VH), a light chain constant region (CL), and the first constant region of the heavy chain (CH1); (ii) an Fd fragment consisting of the VH and CH1 domain; (iii) an Fv fragment consisting of the VL and VH domains of a single antibody; (iv) a dAb fragment consisting of the VH domain (Ward E S et al., Nature 341:544-546 (1989)); (v) an isolated CDR region; (vi) F(ab′)2 fragment, which is a bivalent fragment including two linked Fab fragments; (vii) scFv, which is single chain Fv molecules joined by a peptide linker joining the VH and VL domains to form an antigen binding site; (viii) bispecific single chain Fv dimers (PCT/US92/09965); and (ix) diabody, which is a multivalent or multispecific fragment produced by gene fusion (WO94/13804), and the like.

In the present disclosure, the terms “gas vesicles (GVs)” and “gas vesicle nanoparticles (GVNPs)” refer to the same and may be used interchangeably.

In one aspect, the present disclosure relates to the recombinant gas vesicle nanoparticle prepared by the method of the present disclosure.

In one embodiment, the recombinant gas vesicle nanoparticle prepared by the method of the present disclosure may be used for various purposes depending on the full length of GvpC or an exogenous protein fused with a fragment thereof. For example, when fused with an antigen including epitopes of a specific virus, the recombinant gas vesicle nanoparticle may be used as a vaccine composition. In this case, it is easy to administer and has the effect of increasing stability.

In one embodiment, the recombinant gas vesicle nanoparticle prepared by the method of the present disclosure may include a recombinant protein GvpC-GB in which the full-length or fragment of GvpC and an IgG-binding (GB) domain are fused as an exogenous recombinant protein. In this case, it may be an antibody-loaded recombinant gas vesicle nanoparticle in which at least one antibody is bound to the recombinant GVNP complex.

In one embodiment, the antibody may be a polyclonal antibody, a monoclonal antibody, a minibody, a domain antibody, a bispecific antibody, an antibody mimic, a chimeric antibody, an antibody conjugate, a human antibody or a humanized antibody, or a fragment thereof. The fragment may be any one selected from the group consisting of Fab, Fd, Fab′, dAb, F(ab′), F(ab′)2, single chain fragment variable (scFv), Fv, single chain antibody, Fv dimer, complementarity determining region fragment, humanized antibody, a chimeric antibody and a diabody.

In one embodiment, the antibody may be one which binds to at least one target antigen selected from the group consisting of 17-1A antigen, GD3 ganglioside R24, EGFRvIII, PSMA, PSCA, HLA-DR, EpCAM, MUC1 core protein, aberrant glycosylation MUC1, a fibronectin variant containing an ED-B domain, HER2/neu, carcino-embryonic antigen (CEA), gastrin-releasing peptide (GRP) receptor antigen, mucine antigen, epidermal growth factor receptor (EGF-R), HER3, HER4, MAGE antigen, SART antigen, MUC1 antigen, c-erb-2 antigen, TAG 72, carbonic anhydrase IX, alpha-fetoprotein, A3, antigen specific for A33 antibody, Ba 733, BrE3-antigen, CA125, CD1, CD1a, CD3, CD5, CD15, CD16, CD19, CD20, CD21, CD22, CD23, CD25, CD30, CD33, CD38, CD40, CD45, CD52, CD74, CD79a, CD80, CD138, colon-specific antigen-p (CSAp), CSAp, EGP-1, EGP-2, Ep-CAM, FIt-1, Flt-3, folate receptor, HLA-DR, human chorionic gonadotropin (HCG) and its subunits, hypoxia inducible factor (HIF-1), Ia, IL-2, IL-6, IL-8, insulin-like growth factor-1 (IGF-1), KC4-antigen, KS-1-antigen, KS1-4, Le-Y, macrophage migration inhibitory factor (MIF), MAGE, MUC1, MUC2, MUC3, MUC4, NCA66, NCA95, NCA90, antigen specific for PAM-4 antibody, placental growth factor, p53, prostatic acid phosphatase, PSA, RS5, S1OO, TAC, tenascin, TRAIL receptors, Tn antigen, Thomson-Friedenreich antigens, tumor necrosis antigens, VEGF, ED-B fibronectin, an angiogenesis marker, an oncogene marker, an oncogene product, a cell surface antigen and an autoantigen.

In one embodiment, the present disclosure relates to an isolated nucleic acid molecule encoding a recombinant gas vesicle nanoparticle, a vector including the same, and a host cell transformed therewith.

Nucleic acid molecules of the present disclosure may be isolated or recombined and include single-stranded and double-stranded forms of DNA and RNA as well as corresponding complementary sequences. In the case that the isolated nucleic acid is a nucleic acid isolated from a naturally occurring source, the nucleic acid is a nucleic acid that has been separated from surrounding genetic sequences present in the genome of the individual from which it was isolated. In the case of a nucleic acid synthesized enzymatically or chemically from a template, such as a PCR product, a cDNA molecule, or an oligonucleotide, a nucleic acid resulting from such a procedure can be understood as an isolated nucleic acid molecule. An isolated nucleic acid molecule refers to a nucleic acid molecule in the form of separate fragments or as a component of a larger nucleic acid construct. A nucleic acid is operably linked when arranged in a functional relationship with another nucleic acid sequence. For example, the DNA of a presequence or secretory leader is operably linked to the DNA of the polypeptide when expressed as a preprotein, which is a presecretory polypeptide. A promoter or an enhancer affecting the transcription of the polypeptide sequence is operably linked to a coding sequence, or a ribosome-binding site is operably linked to a coding sequence when it is arranged such that translation is promoted. Generally, the term “operably linked” means that DNA sequences to be linked are located adjacent to each other. In the case of secretory leaders, the term “operably linked” means that the secretory leaders are present adjacent to each other in the same leading frame. However, an enhancer is not necessarily contiguous. The linkage is performed by ligation at a convenient restriction enzyme site. In the case where this site does not exist, a synthetic oligonucleotide adaptor or a linker is used according to a suitable method known in the art.

In the isolated nucleic acid molecule encoding the recombinant gas vesicle nanoparticle of the present disclosure, in consideration of the degeneracy of codons or codons preferred in the organism in which the recombinant gas vesicle nanoparticle is to be expressed, various modifications can be made to the coding area within the range that does not change the amino acid sequence of the antibody expressed from the coding region and various modifications or changes can be made within the range that does not affect the expression of genes even in parts other than the coding region, and those skilled in the art will understand that the modified gene is also included in the scope of the present disclosure. In other words, as long as the nucleic acid molecule of the present disclosure encodes a protein having equivalent activity, one or more nucleic acid bases may be mutated by substitution, deletion, insertion, or a combination thereof, and these are also included within the scope of the present disclosure. The sequence of such a nucleic acid molecule may be single-stranded or double-stranded and may be a DNA molecule or an RNA (mRNA) molecule.

An isolated nucleic acid molecule encoding a recombinant gas vesicle nanoparticle of the present disclosure according to the present disclosure can be inserted into an expression vector for protein expression. Expression vectors typically include a protein operably linked, that is, placed in a functional relationship, with control or regulatory sequences, selectable markers, any fusion partners, and/or additional elements. The recombinant gas vesicle nanoparticle of the present disclosure may be produced by a method in which a host cell transformed with a nucleic acid, preferably an expression vector containing an isolated nucleic acid molecule encoding a recombinant gas vesicle nanoparticle of the present disclosure is cultured to induce protein expression in appropriate conditions. A wide variety of appropriate host cells may be used, including but not limited to mammalian cells, bacteria, insect cells, and yeast. The methods of introducing exogenous nucleic acid into host cells are well known in the art and will vary with the host cell used. Preferably, E. coli, which has a high industrial use value due to low production cost, can be produced as a host cell.

As used herein, the term “expression vector” refers to a vector containing a nucleic acid sequence coding at least part of a gene product capable of being transcribed. In some cases, RNA molecules are then translated into a protein, polypeptide, or peptide. Expression vectors can contain a variety of control sequences. In addition to control sequences that govern transcription and translation, vectors and expression vectors may contain nucleic acid sequences that serve other functions as well.

As used herein, the term “host cell” includes eukaryotes, prokaryotes and archaea and refers to any transgenic organism that is capable of replicating the vector or expressing the gene encoded by the vector. The host cell may be transfected or transformed by the vector. The transfection or transformation refers to a process for transferring or introducing the exogenous nucleic acid molecule into the host cell.

In one aspect, the present disclosure relates to a pharmaceutical composition for preventing or treating a disease or disorder, the composition including the antibody-loaded recombinant gas vesicle nanoparticle as an active ingredient, in which the disease or disorder is selected from the group consisting of cancer, autoimmune disease, neurodegenerative disease, Alzheimer's disease, metabolic disease, cardiovascular disease, atherosclerosis, organ transplant rejection, and disease or symptom caused by a fungus, virus, bacteria or parasites.

As used herein, the term “prevention” refers to any action that suppresses or delays the occurrence, spread and recurrence of a disease or disorder by administration of a composition according to the present disclosure.

As used herein, the term “treatment” refers to any action that improves or advantageously changes the symptoms of a disease or disorder and its complications by administration of a composition according to the present disclosure. Those of ordinary skill in the art to which the present disclosure belongs can know the exact standard of the disease for which the composition of the present application is effective by referring to the data presented in the Korean Academy of Medical Sciences, etc., and determine the degree of enhancement, improvement and treatment.

As used herein, the term “therapeutically effective amount” used in combination with an active ingredient means an amount effective to prevent or treat a disease or disorder and a therapeutically effective amount of the composition of the present disclosure may vary depending on several factors, such as the method of administration, the target site, the condition of the patient, etc. Therefore, when used in the human body, the dosage should be determined as an appropriate amount in consideration of both safety and efficiency. It is also possible to estimate the amount used in humans from the effective amount determined through animal experiments. These considerations in determining effective amounts are described for example, in Hardman and Limbird, eds., Goodman and Gilman's The Pharmacological Basis of Therapeutics, 10th ed.(2001), Pergamon Press; and E. W. Martin ed., Remington's Pharmaceutical Sciences, 18th ed.(1990), Mack Publishing Co.

The pharmaceutical composition of the present disclosure is administered in a pharmaceutically effective amount. As used herein, the term “pharmaceutically effective amount” means an amount sufficient to treat a disease at a reasonable benefit/risk ratio applicable to medical treatment and not to cause side effects, and the effective dose level may depend on the patient's health condition, disease or disorder type, disease or disorder severity, drug activity, drug sensitivity, administration method, administration time, administration route and excretion rate, treatment period, combination, factors including co-administered drugs, and other factors well known in the medical arts. The composition according to the present disclosure may be administered as an individual therapeutic agent or in combination with other therapeutic agents, sequentially or concurrently with conventional therapeutic agents, and may be administered singly or in multiple doses. It is important to take into account all of the above factors and to administer the amount in which the maximum effect can be obtained in a minimal amount without side effects, which can be easily determined by those skilled in the art.

The pharmaceutical composition of the present disclosure may include a carrier, diluent, excipient, or a combination of two or more commonly used in biological agents. As used herein, the term “pharmaceutically acceptable” refers to exhibiting properties that are not toxic to cells or humans exposed to the composition. The carrier is not particularly limited as long as it is suitable for in vivo delivery of the composition, and for example, the compound described in Merck Index, 13th ed., Merck & Co. Inc., saline, sterile water, Ringer's solution, buffered saline, dextrose solution, maltodextrin solution, glycerol, ethanol, and one or more of these components may be mixed and used. If necessary, other conventional additives such as antioxidant, buffer, and bacteriostat may be added. In addition, diluent, dispersant, surfactant, binder and lubricant may be further added to formulate into an injectable formulation such as an aqueous solution, suspension, emulsion, etc., pill, capsule, granule or tablet. Furthermore, it may be preferably formulated according to each disease or component using an appropriate method in the art or a method disclosed in Remington's Pharmaceutical Science (Mack Publishing Company, Easton Pa., 18th, 1990).

In one embodiment, the pharmaceutical composition may be one or more formulations selected from the group consisting of oral formulations, topical formulations, suppositories, sterile injection solutions, and sprays, and may be more preferably oral or injection formulations.

As used herein, the term “administration” means providing a given substance to an individual or patient by any suitable method. Parenteral administration (for example, intravenous, subcutaneous, intraperitoneal, or topical application as an injection formulation) or oral administration may be performed depending on the desired method. The dosage varies depending on the patient's weight, age, sex, health condition, diet, administration time, administration method, excretion rate, and severity of disease. Liquid formulations for oral administration of the composition of the present disclosure include suspensions, internal solutions, emulsions, syrups, etc. and may include in addition to water and liquid paraffin, which are commonly used simple diluents, various excipients such as wetting agents, sweeteners, fragrances, preservatives and the like. Formulations for parenteral administration include sterile aqueous solutions, non-aqueous solvents, suspensions, emulsions, freeze-dried preparations, suppositories, and the like. The pharmaceutical composition of the present disclosure may be administered by any device capable of delivering an active substance to a target cell. Preferred administration methods and formulations include intravenous injections, subcutaneous injections, intradermal injections, intramuscular injections, drip injections, and the like. Injections may be prepared using aqueous solvents such as physiological saline solution and Ringer's solution, and non-aqueous solvents such as vegetable oil, higher fatty acid esters (e.g., ethyl oleate), and alcohols (e.g., ethanol, benzyl alcohol, propylene glycol, glycerin, etc.) and may include pharmaceutical carriers such as stabilizers for preventing deterioration (e.g., ascorbic acid, sodium hydrogen sulfite, sodium pyrosulfite, BHA, tocopherol, EDTA, etc.), emulsifiers, buffers for pH adjustment, preservatives to inhibit the growth of microorganisms (e.g., phenylmercuric nitrate, thimerosal, benzalkonium chloride, phenol, cresol, benzyl alcohol, etc.).

As used herein, the term “individual” means all animals including monkeys, cattle, horses, sheep, pigs, chickens, fish (cultured fish), turkeys, quails, cats, dogs, mice, bats, camels, rats, rabbits, or guinea pigs, in addition to human. The term “sample” may be droplets, sputum, whole blood, plasma, serum, urine, or saliva isolated therefrom.

The pharmaceutical composition of the present disclosure may further include a pharmaceutically acceptable additive, in which the pharmaceutically acceptable additive may include starch, gelatinized starch, microcrystalline cellulose, lactose, povidone, colloidal silicon dioxide, hydrogen phosphate calcium, lactose, mannitol, taffy, arabic rubber, pregelatinized starch, corn starch, powdered cellulose, hydroxypropyl cellulose, Opadry, sodium starch glycolate, lead carnauba, synthetic aluminum silicate, stearic acid, magnesium stearate, aluminum stearate, calcium stearate, sucrose, dextrose, sorbitol and talc, etc. The pharmaceutically acceptable additive according to the present disclosure is preferably included in an amount of 0.1 parts by weight to 90 parts by weight with respect to the composition but is not limited thereto.

The present disclosure is described in more detail through the following examples. However, the following examples are only intended to materialize the content of the present disclosure, and thus the present disclosure is not limited thereto.

### Example 1. Construction of GvpC-IgG-Binding Domain (GB) Fusion Protein

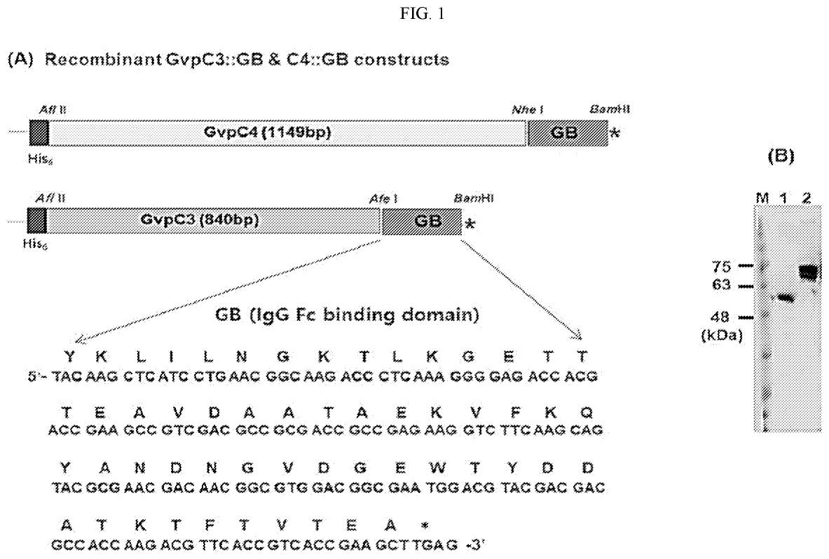

Two versions of a GvpC-Streptococcal IgG-binding (GB) domain fusion protein containing GvpC of different lengths were constructed to include GvpC4 GB containing full-length GvpC and GvpC3 GB having a C-terminal acid tail deleted. Specifically, the streptococcal protein GB1 domain was selected for expression as a fusion protein, and 4 oligonucleotides containing 54 amino acids of the IgG-binding (GB) domain and sequences corresponding to the restriction sites of AfeI and BamHI at both ends (Table 1) were annealed to construct two fusion genes encoding GvpC4 GB and GvpC3 GB. These were inserted into E. coli expression plasmid pET19b to prepare constructs of expression plasmids pET19b-GvpC3 GB and pET19b-GvpC4 GB (a) of FIG. 1). The prepared plasmids were transformed and induced into E. coli BL21 (DE3), followed by expression, and then the fusion proteins were purified by affinity column chromatography. The purified fusion proteins were confirmed by identifying bands of 60 kDa (GvpC3 GB) and 70 kDa (GvpC4 GB) by immunoblot analysis using an anti-rabbit IgG antibody (b) of FIG. 1). For reference, the actual molecular weight of GvpC4 GB is 48 kDa and that of GvpC3 GB is 37 kDa, but the molecular weight fluctuation is a phenomenon previously observed because it is a highly acidic Halobacterium protein (Halladay et al. 1993; Shukla and DasSarma 2004).

TABLE 1

| Primer | |
|---|---|
| name | Sequence (5′->3′) |
| GB1F | GCT TAC AAG CTC ATC CTG AAC |
| | GGC AAG ACC CTC AAA GGG |
| | GAG ACC ACG ACC GAA GCC GTC |
| | GAC GCC GCG ACC GCC |
| | GAG |
| GB2F | AAG GTC TTC AAG CAG TAC GCG |
| | AAC GAC AAC GGC GTG |
| | GAC GGC GAA TGG ACG TAC GAC |
| | GAC GCC ACC AAG ACG |
| | TTC ACC GTC ACC GAA GCT TGA |
| | G |
| GB2R | GATCC TCA AGC TTC GGT GAC |
| | GGT GAA CGT CTT GGT GGC |
| | GTC GTC GTA CGT CCA TTC GCC |
| | GTC CAC GCC GTT GTC GTT |
| | CGC |
| GB1R | GTA CTG CTT GAA GAC CTT CTC |
| | GGC GGT CGC GGC GTC GAC |
| | GGC TTC GGT CGT GGT CTC CCC |
| | TTT GAG GGT CTT GCC GTT |
| | CAG GAT GAG CTT GTA AGC |

### Example 2. GVNP Purification and GvpC Removal

Halobacterium sp. NRC-1 (ATCC 700922/JCM11081) was cultured in CM⁺ medium and then dispensed on CM⁺ agar plates. It was cultured at 37° C. for 3 weeks until pink turbid lawns were formed (DasSarma et al. 1995). After that, 5 ml of 1× PBS containing 1 mM MgSO₄ and 1 mg/ml of DNAse I (Sigma Aldrich) (137 mM NaCl, 2.7 mM KCl, 10 mM sodium phosphate dibasic, and 2 mM potassium phosphate monobasic (pH 7.2)) was added in the cluster of Halobacterium sp. NRC-1 to lyse the cells, followed by incubation at 37° C. for 3 hours. The lysate was centrifuged at 60×g for 12 hours to collect intact buoyant GVNPs, resuspended in 2 to 3 times the volume of 1× PBS, followed by centrifugation. Then, the accelerated flotation process was performed four more times until purified GVNPs as a milky suspension were obtained. After treating purified GVNP with 5 M urea for 2 hours, dialysis was performed to prepare GVNP (DGvpC) from which GvpC was removed.

### Example 3. Preparation of GVNP Complex to which Fusion Protein is Bound

3-1. Confirmation of GVNP Complex Formation According to Salt Concentration

The binding of the exogenous GvpC fusion protein to Haloarchaeal GVNPs was confirmed at various salt concentrations.

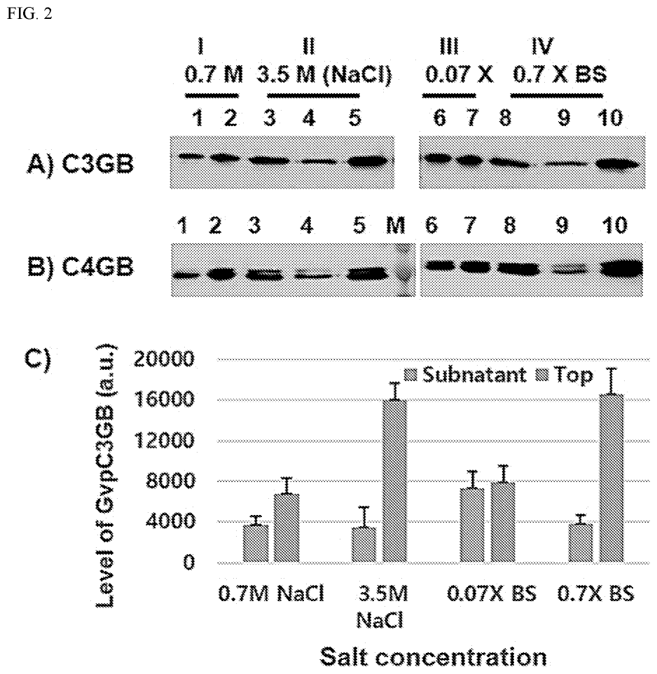

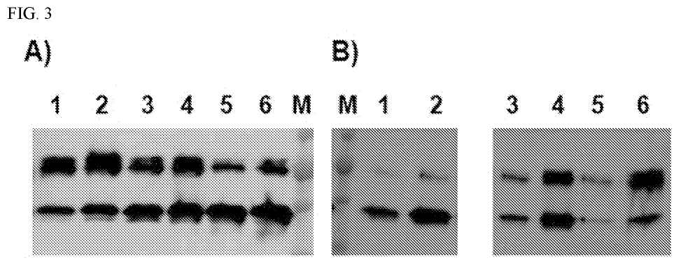

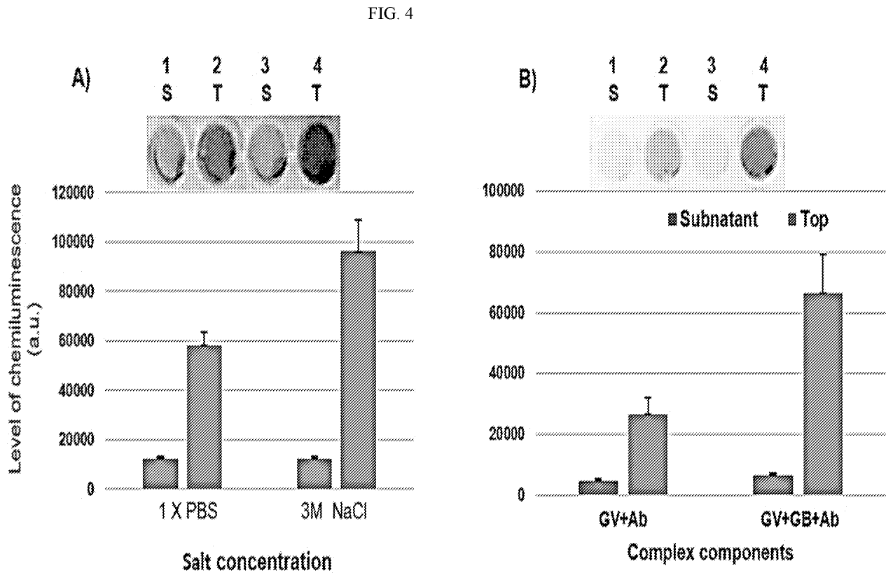

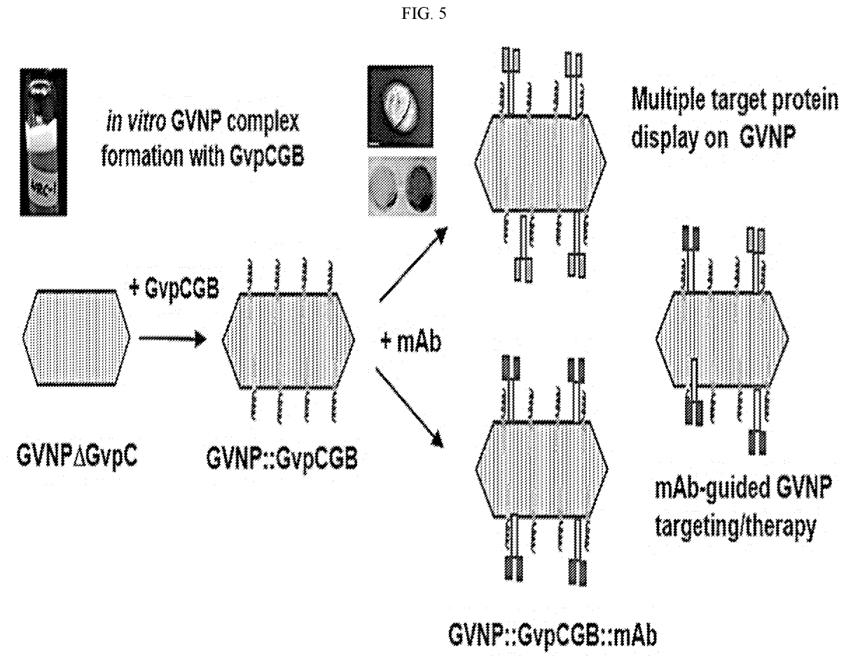

Specifically, the fusion proteins GvpC4 GB and GvpC3 GB, respectively, prepared in Example 1 were mixed with GVNP (DGvpC) prepared in Example 2. Each of them was incubated at 25° C. for 1.5 hours under a condition of 0.7 M NaCl, 3.5 M NaCl, 0.07× BS (Basal Salts) (250 g NaCl, 20 g MgSO₄7H₂O, 2 g KCl, and 3 g Na₃C₆H₅O₇2H₂O) or 0.7× BS. Then, the centrifugation was performed at 300× g for 30 minutes to separate 2/3 volume of the subnatant and the remaining GVNP-containing top/floating layers. To compare the levels of recombinant GvpCGB in the separated GVNP-containing top layer and subnatant, each fraction was mixed with the same volume of 2× SDS-PAGE gel loading buffer containing 100 mM Tris-Cl (pH 6.8), 4% (w/v) SDS, 20% glycerol, and 200 mM β-mercaptoethanol. They were heated for 4 minutes, and then SDS-PAGE and immunoblot analysis were performed, and quantification was performed with ImageJ. As a result, it was confirmed that GvpC fusion protein, GvpCGBs, was significantly higher in GVNP-containing top layer compared to the subnatant, confirming that both GvpC3 GB and GvpC4 GB could form a complex with purified wild-type GVNPs. In addition, the GVNP complex formation level was shown to increase at a higher salt concentration (FIG. 2), suggesting that the binding of recombinant GvpC to the GVNP platform is easier under high salt conditions.

3-2. Confirmation of GVNP Complex Formation According to Type of Fusion Protein

To confirm competitive binding with Haloarchaeal GVNPs with or without the C-terminal region of GvpCGB, an exogenous GvpC fusion protein, the degree of binding was confirmed by mixing the two types of fusion proteins GvpC3 GB and GvpC4 GB with GVNP while changing the amount at various ratios. Specifically, the fusion proteins GvpC3 GB and GvpC4 GB prepared in Example 1 were mixed in various ratios (5:1, 1:1 and 1:5), and then mixed with GVNP (ΔGvpC) prepared in Example 2, respectively. They were incubated at 25° C. for 1.5 hours and centrifuged at 300×g for 30 minutes to separate 2/3 volume of the subnatant and the remaining GVNP-containing top fraction (top layer). In order to compare the levels of recombinant GvpCGB in separated GVNP-containing subnatant and floating layer according to the ratio of GvpC3 GB and GvpC4 GB, SDS-PAGE and immunoblot analysis were performed as in Example 3-1 above. As a result, when binding was performed with excessive recombinant GvpCGBs compared to GVNPs, the levels of GvpC3 GB and GvpC4 GB detected in the subnatant and the top layer were similar to each other at a predetermined ratio (a) of FIG. 3). The GVNP-containing top fraction isolated above was collected, diluted in 1× PBS containing 5 times the volume of 3 M NaCl, and centrifuged twice at low speed, and immunoblot analysis was performed. Here, lane 2 of b) of FIG. 3 is a top layer of a mixture of GvpC3 GB and GvpC4 GB at a molecular weight of 5:1; lane 4 is a top layer of a mixture of GvpC3 GB and GvpC4 GB at a molecular weight of 1:1; and lane 6 is a top layer of a mixture of GvpC3 GB and GvpC4 GB at a molecular weight of 1:5, and the subnatant obtained by diluting them again and centrifuging them is lanes 1, 3 and 5, and the top layer is lanes 2, 4 and 6.

As a result, more fusion proteins were detected in the GVNP-containing top layer (2, 4, and 6) than in the subnatant (1, 3, and 5), indicating that a significant portion of GvpC3 GB remained bound to GVNPs (b) of FIG. 3). The ratio of GvpC3 GB fusion protein and GvpC4 GB fusion protein (1:5 to 5:1) detected in the top layer containing GVNP in each experiment was found to be similar to the ratio of the original mixture. These results indicate that there is no preferential binding according to the GvpC length of the fusion protein, and that the acidic amino acid-rich tail of C-terminal of GvpCGB does not increase binding to GVNP.

### Example 4. Confirmation of Functionality of GVNP Complex to which Recombinant GvpCGB is Bound

Whether the GVNP complex displaying the IgG-binding (GB) domain prepared in Example 3 was functional was confirmed by binding to an HRP-linked anti-rabbit IgG antibody. For binding analysis, a GvpC3 GB-GVNP complex was prepared as in the above example in 1× PBS or 3 M NaCl PBS buffer using GvpC3 GB fusion protein and excess GVNPs. After collecting the GVNP-containing top layer, they were incubated with HRP-linked secondary antibody at the same salt concentration and then centrifuged. Thereafter, the level of GvpC3 GB in each fraction was confirmed by a chemiluminescence reaction catalyzed by an HRP-linked antibody (a) of FIG. 4). As a result, chemiluminescence was significantly higher in the GVNP-containing top layer (2 and 4 in a) of FIG. 4) than in the subnatant (1 and 3 in a) of FIG. 4), confirming that GvpC3 GB was bound to the GVNP complex (a) of FIG. 4). Further, the level of chemiluminescence in the presence of 3 M NaCl (4 in a) of FIG. 4) was higher than that of 1× PBS (2 in a) of FIG. 4), confirming that GvpC3 GB and GVNP complex formation was promoted at a high salt concentration (a) of FIG. 4). Further, in order to confirm the level of chemiluminescence due to non-specific binding of the HRP-linked antibody to the components of wild-type GVNPs, binding analysis was performed according to the presence or absence of the fusion protein. The experimental results showed that the HRP-linked antibody had a weak non-specific binding to GVNP in the absence of GvpC3 GB (1 and 2 of b) of FIG. 4), but showed a much higher level of chemiluminescence in the presence of GvpC3 GB. The experimental results showed that the GvpC3 GB fusion protein binds to GVNPs and maintains the function of GB binding to IgG on the outer surface of the GVNP complex (b) of FIG. 4).

From the foregoing, it will be appreciated that various embodiments of the present disclosure have been described herein for purposes of illustration, and that various modifications may be made without departing from the scope and spirit of the present disclosure. Accordingly, the various embodiments disclosed herein are not intended to be limiting, with the true scope and spirit being indicated by the following claims.

What is claimed is:

1. A method for preparing a recombinant gas vesicle nanoparticle (GVNP), the method comprising steps of:
a) culturing Halobacterium sp.;
b) collecting a gas vesicle (GV);
c) removing GvpC from the gas vesicle;
d) preparing a recombinant protein as an exogenous recombinant protein in which a full length or fragment of GvpC and an exogenous protein are fused; and
e) reacting the gas vesicle from which GvpC has been removed with the exogenous recombinant protein to form a recombinant GVNP complex.

2. The method of claim 1, wherein the Halobacterium sp. is Halobacterium sp NRC-1.

3. The method of claim 1, wherein the GvpC is removed by treatment with urea and dialysis in step c).

4. The method of claim 1, wherein the exogenous protein is an antigen.

5. The method of claim 1, wherein the exogenous protein is an IgG-binding (GB) domain.

6. The method of claim 5, further comprising a step of binding at least one antibody to the recombinant GVNP complex.

7. The method of claim 1, wherein the exogenous recombinant protein is expressed and purified in Escherichia coli.

8. A recombinant gas vesicle nanoparticle prepared by the method of claim 1.

9. A vaccine composition comprising the recombinant gas vesicle nanoparticle prepared by the method of claim 4.

10. An antibody-loaded recombinant gas vesicle nanoparticle prepared by the method of claim 6.

11. The antibody-loaded recombinant gas vesicle nanoparticle of claim 10, wherein the antibody is a polyclonal antibody, a monoclonal antibody, a minibody, a domain antibody, a bispecific antibody, an antibody mimic, a chimeric antibody, an antibody conjugate, a human antibody or a humanized antibody, or a fragment thereof.

12. The antibody-loaded recombinant gas vesicle nanoparticle of claim 10, wherein the antibody binds to at least one target antigen selected from the group consisting of 17-1A antigen, GD3 ganglioside R24, EGFRvIII, PSMA, PSCA, HLA-DR, EpCAM, MUC1 core protein, aberrant glycosylation MUC1, a fibronectin variant containing an ED-B domain, HER2/neu, carcino-embryonic antigen (CEA), gastrin-releasing peptide (GRP) receptor antigen, mucine antigen, epidermal growth factor receptor (EGF-R), HER3, HER4, MAGE antigen, SART antigen, MUC1 antigen, c-erb-2 antigen, TAG 72, carbonic anhydrase IX, alpha-fetoprotein, A3, antigen specific for A33 antibody, Ba 733, BrE3-antigen, CA125, CD1, CD1a, CD3, CD5, CD15, CD16, CD19, CD20, CD21, CD22, CD23, CD25, CD30, CD33, CD38, CD40, CD45, CD52, CD74, CD79a, CD80, CD138, colon-specific antigen-p (CSAp), CSAp, EGP-1, EGP-2, Ep-CAM, FIt-1, Flt-3, folate receptor, human chorionic gonadotropin (HCG) and its subunits, hypoxia inducible factor (HIF-1), Ia, IL-2, IL-6, IL-8, insulin-like growth factor-1 (IGF-1), KC4-antigen, KS-1-antigen, KS1-4, Le-Y, macrophage migration inhibitory factor (MIF), MAGE, MUC1, MUC2, MUC3, MUC4, NCA66, NCA95, NCA90, antigen specific for PAM-4 antibody, placental growth factor, p53, prostatic acid phosphatase, PSA, RS5, S1OO, TAC, tenascin, TRAIL receptors, Tn antigen, Thomson-Friedenreich antigens, tumor necrosis antigens, VEGF, ED-B fibronectin, an angiogenesis marker, an oncogene marker, an oncogene product, a cell surface antigen and an autoantigen.

13. A pharmaceutical composition for preventing or treating a disease or disorder, the composition comprising the antibody-loaded recombinant gas vesicle nanoparticle of claim 10 as an active ingredient, wherein the disease or disorder is selected from the group consisting of cancer, autoimmune disease, neurodegenerative disease, Alzheimer's disease, metabolic disease, cardiovascular disease, atherosclerosis, organ transplant rejection, and disease or symptom caused by fungus, virus, bacteria or parasites.


SEQUENCE LISTING (10 sequences)

SEQ ID NO: 1 - protein, 382 aa, unidentified
MSVTDKRDEMSTARDKFAESQQEFESYADEFAADITAKQDDVSDLVDAITDFQAEMTNTTDAFHTYGDEFAAEVDHLRADIDAQRDVIREMQDAFEAYADIFATDIADKQDIGNLQAAIEALRTEMNSTHGAFEAYADDFAADVAALRDISDLVAAIDDFQEEFIAVQDAFDNYAGDFDAEIDQLHAAIADQHDSFDATADAFAEYRDEFYRIEVEALLEAINDFQQDIGDFRAEFETTEDAFVAFARDFYGHEITAEEGAAEAEAEPVEADADVEAEAEVSPDEAGGESAGTEEEETEPAEVETAAPEVEGSPADTADEAEDTEAEEETEEEAPEDMVQCRVCGEYYQAITEPHLQTHDMTIQEYRDEYGEDVPLRPDDKT

SEQ ID NO: 2 - protein, 284 aa, unidentified
MSVTDKRDEMSTARDKFAESQQEFESYADEFAADITAKQDDVSDLVDAITDFQAEMTNTTDAFHTYGDEFAAEVDHLRADIDAQRDVIREMQDAFEAYADIFATDIADKQDIGNLQAAIEALRTEMNSTHGAFEAYADDFAADVAALRDISDLVAAIDDFQEEFIAVQDAFDNYAGDFDAEIDQLHAAIADQHDSFDATADAFAEYRDEFYRIEVEALLEAINDFQQDIGDFRAEFETTEDAFVAFARDFYGHEITAEEGAAEAEAEPVEADADVEAEAEPRSA

SEQ ID NO: 3 - protein, 55 aa, unidentified
YKLILNGKTLKGETTTEAVDAATAEKVFKQYANDNGVDGEWTYDDATKTFTVTEA

SEQ ID NO: 4 - protein, 450 aa, unidentified
MHHHHHHLKMSVTDKRDEMSTARDKFAESQQEFESYADEFAADITAKQDDVSDLVDAITDFQAEMTNTTDAFHTYGDEFAAEVDHLRADIDAQRDVIREMQDAFEAYADIFATDIADKQDIGNLQAAIEALRTEMNSTHGAFEAYADDFAADVAALRDISDLVAAIDDFQEEFIAVQDAFDNYAGDFDAEIDQLHAAIADQHDSFDATADAFAEYRDEFYRIEVEALLEAINDFQQDIGDFRAEFETTEDAFVAFARDFYGHEITAEEGAAEAEAEPVEADADVEAEAEVSPDEAGGESAGTEEEETEPAEVETAAPEVEGSPADTADEAEDTEAEEETEEEAPEDMVQCRVCGEYYQAITEPHLQTHDMTIQEYRDEYGEDVPLRPDDKTARSAYKLILNGKTLKGETTTEAVDAATAEKVFKQYANDNGVDGEWTYDDATKTFTVTEA

SEQ ID NO: 5 - protein, 348 aa, unidentified
MHHHHHHLKMSVTDKRDEMSTARDKFAESQQEFESYADEFAADITAKQDDVSDLVDAITDFQAEMTNTTDAFHTYGDEFAAEVDHLRADIDAQRDVIREMQDAFEAYADIFATDIADKQDIGNLQAAIEALRTEMNSTHGAFEAYADDFAADVAALRDISDLVAAIDDFQEEFIAVQDAFDNYAGDFDAEIDQLHAAIADQHDSFDATADAFAEYRDEFYRIEVEALLEAINDFQQDIGDFRAEFETTEDAFVAFARDFYGHEITAEEGAAEAEAEPVEADADVEAEAEPRSAYKLILNGKTLKGETTTEAVDAATAEKVFKQYANDNGVDGEWTYDDATKTFTVTEA

SEQ ID NO: 6 - DNA, 1149 bp, unidentified
  source 1..1149: mol_type = unassigned DNA
atgagtgtcacagacaaacgcgacgagatgagtactgcccgcgataagttcgcagaatcacagcaggagttcgaatcatacgctgacgagtttgcagccgatatcacggcaaagcaagacgatgtcagcgaccttgtcgatgcgatcaccgacttccaggcggagatgaccaacacgacggatgcatttcacacatatggtgacgagttcgccgctgaggttgaccacctccgtgccgatattgacgcccagcgggacgtgatccgtgagatgcaggatgcgttcgaggcatatgctgacatcttcgctacagatatcgcagacaaacaagatatcggcaatcttcaggctgcgattgaggcgctccgaacagagatgaactcaacccacggggcattcgaagcatatgcggacgacttcgcagccgatgtcgctgcgctccgtgatatatctgatctggttgcagcaatcgacgacttccaagaggaattcatcgccgtgcaggacgcatttgacaactacgctggtgacttcgatgcggagatcgaccagctccacgctgccatcgctgaccagcacgacagcttcgacgctaccgcggacgccttcgcagagtaccgagatgagttctatcgcatagaggtggaagcactgcttgaggcgatcaacgacttccagcaggacatcggtgacttccgagcggagtttgaaacgactgaggacgcgttcgttgccttcgcccgtgacttctatggccacgagatcacggccgaggaaggcgccgccgaagcggaagccgaacccgtcgaggctgacgcggacgtcgaagcggaagcagaagtctctccagacgaagctggcggagaatccgccggtaccgaggaagaagagacagagccggccgaggtggaaacagcggctccagaagtagaggggagtcctgcggacacggcagacgaagcggaagatacggaagcagaggaggagacagaggaagaggcaccggaagacatggtgcagtgccgggtgtgcggcgaatactatcaggccatcacggagccccatctccagacccatgatatgacgattcaggagtaccgcgacgagtacggtgaggatgtcccccttcggccggatgataaaacatga

SEQ ID NO: 7 - DNA, 852 bp, unidentified
  source 1..852: mol_type = unassigned DNA
atgagtgtcacagacaaacgcgacgagatgagtactgcccgcgataagttcgcagaatcacagcaggagttcgaatcatacgctgacgagtttgcagccgatatcacggcaaagcaagacgatgtcagcgaccttgtcgatgcgatcaccgacttccaggcggagatgaccaacacgacggatgcatttcacacatatggtgacgagttcgccgctgaggttgaccacctccgtgccgatattgacgcccagcgggacgtgatccgtgagatgcaggatgcgttcgaggcatatgctgacatcttcgctacagatatcgcagacaaacaagatatcggcaatcttcaggctgcgattgaggcgctccgaacagagatgaactcaacccacggggcattcgaagcatatgcggacgacttcgcagccgatgtcgctgcgctccgtgatatatctgatctggttgcagcaatcgacgacttccaagaggaattcatcgccgtgcaggacgcatttgacaactacgctggtgacttcgatgcggagatcgaccagctccacgctgccatcgctgaccagcacgacagcttcgacgctaccgcggacgccttcgcagagtaccgagatgagttctatcgcatagaggtggaagcactgcttgaggcgatcaacgacttccagcaggacatcggtgacttccgagcggagtttgaaacgactgaggacgcgttcgttgccttcgcccgtgacttctatggccacgagatcacggccgaggaaggcgccgccgaagcggaagccgaacccgtcgaggctgacgcggacgtcgaagcggaagcagaacctaggagctga

SEQ ID NO: 8 - DNA, 168 bp, unidentified
  source 1..168: mol_type = unassigned DNA
tacaagctcatcctgaacggcaagaccctcaaaggggagaccacgaccgaagccgtcgacgccgcgaccgccgagaaggtcttcaagcagtacgcgaacgacaacggcgtggacggcgaatggacgtacgacgacgccaccaagacgttcaccgtcaccgaagcttga

SEQ ID NO: 9 - DNA, 1353 bp, unidentified
  source 1..1353: mol_type = unassigned DNA
atgcaccaccaccaccaccaccttaagatgagtgtcacagacaaacgcgacgagatgagtactgcccgcgataagttcgcagaatcacagcaggagttcgaatcatacgctgacgagtttgcagccgatatcacggcaaagcaagacgatgtcagcgaccttgtcgatgcgatcaccgacttccaggcggagatgaccaacacgacggatgcatttcacacatatggtgacgagttcgccgctgaggttgaccacctccgtgccgatattgacgcccagcgggacgtgatccgtgagatgcaggatgcgttcgaggcatatgctgacatcttcgctacagatatcgcagacaaacaagatatcggcaatcttcaggctgcgattgaggcgctccgaacagagatgaactcaacccacggggcattcgaagcatatgcggacgacttcgcagccgatgtcgctgcgctccgtgatatatctgatctggttgcagcaatcgacgacttccaagaggaattcatcgccgtgcaggacgcatttgacaactacgctggtgacttcgatgcggagatcgaccagctccacgctgccatcgctgaccagcacgacagcttcgacgctaccgcggacgccttcgcagagtaccgagatgagttctatcgcatagaggtggaagcactgcttgaggcgatcaacgacttccagcaggacatcggtgacttccgagcggagtttgaaacgactgaggacgcgttcgttgccttcgcccgtgacttctatggccacgagatcacggccgaggaaggcgccgccgaagcggaagccgaacccgtcgaggctgacgcggacgtcgaagcggaagcagaagtctctccagacgaagctggcggagaatccgccggtaccgaggaagaagagacagagccggccgaggtggaaacagcggctccagaagtagaggggagtcctgcggacacggcagacgaagcggaagatacggaagcagaggaggagacagaggaagaggcaccggaagacatggtgcagtgccgggtgtgcggcgaatactatcaggccatcacggagccccatctccagacccatgatatgacgattcaggagtaccgcgacgagtacggtgaggatgtcccccttcggccggatgataaaacagctaggagcgcttacaagctcatcctgaacggcaagaccctcaaaggggagaccacgaccgaagccgtcgacgccgcgaccgccgagaaggtcttcaagcagtacgcgaacgacaacggcgtggacggcgaatggacgtacgacgacgccaccaagacgttcaccgtcaccgaagcttga

SEQ ID NO: 10 - DNA, 1047 bp, unidentified
  source 1..1047: mol_type = unassigned DNA
atgcaccaccaccaccaccaccttaagatgagtgtcacagacaaacgcgacgagatgagtactgcccgcgataagttcgcagaatcacagcaggagttcgaatcatacgctgacgagtttgcagccgatatcacggcaaagcaagacgatgtcagcgaccttgtcgatgcgatcaccgacttccaggcggagatgaccaacacgacggatgcatttcacacatatggtgacgagttcgccgctgaggttgaccacctccgtgccgatattgacgcccagcgggacgtgatccgtgagatgcaggatgcgttcgaggcatatgctgacatcttcgctacagatatcgcagacaaacaagatatcggcaatcttcaggctgcgattgaggcgctccgaacagagatgaactcaacccacggggcattcgaagcatatgcggacgacttcgcagccgatgtcgctgcgctccgtgatatatctgatctggttgcagcaatcgacgacttccaagaggaattcatcgccgtgcaggacgcatttgacaactacgctggtgacttcgatgcggagatcgaccagctccacgctgccatcgctgaccagcacgacagcttcgacgctaccgcggacgccttcgcagagtaccgagatgagttctatcgcatagaggtggaagcactgcttgaggcgatcaacgacttccagcaggacatcggtgacttccgagcggagtttgaaacgactgaggacgcgttcgttgccttcgcccgtgacttctatggccacgagatcacggccgaggaaggcgccgccgaagcggaagccgaacccgtcgaggctgacgcggacgtcgaagcggaagcagaacctaggagcgcttacaagctcatcctgaacggcaagaccctcaaaggggagaccacgaccgaagccgtcgacgccgcgaccgccgagaaggtcttcaagcagtacgcgaacgacaacggcgtggacggcgaatggacgtacgacgacgccaccaagacgttcaccgtcaccgaagcttga